(19) United States
(12) Patent Application Publication
(10) Pub. No.: US 2025/0128060 A1
(43) Pub. Date: Apr. 24, 2025

(54) ELECTROTHERAPY DEVICE COMBINING ELECTROSTIMULATION AND TECARTHERAPY

(71) Applicant: WINBACK GROUP, Biarritz (FR)

(72) Inventors: Christophe BUÉE, Saint Jean de Luz (FR); Gilles JOVÈNE, St Laurent du Var (FR); Sebastien BROUCHET, Beaulieu sur mer (FR)

(73) Assignee: WINBACK GROUP, Biarritz (FR)

(21) Appl. No.: 18/834,505

(22) PCT Filed: Feb. 2, 2023
(86) PCT No.: PCT/EP2023/052602

§ 371 Date: Jul. 30, 2024

(30) Foreign Application Priority Data
Feb. 3, 2022 (FR) FR2200974

(51) Int. Cl. A61N 1/36; H02M 7/48; H02M 7/537

(52) U.S. Cl. CPC A61N 1/36034; H02M 7/4815; H02M 7/537

(57) ABSTRACT

An electrotherapy device including: a first voltage-generating module for generating a first voltage having a first frequency and having a first connection terminal and a second connection terminal, and a second voltage-generating module for generating a second voltage having a second frequency higher than the first frequency and having a third connection terminal and a fourth connection terminal. The device also includes a transmission channel, and a reception channel. The first connection terminal and the third connection terminal are connected to the transmission channel and in that the second connection terminal and the fourth connection terminal are connected to the reception channel. An electronic apparatus for therapeutic or cosmetic use is also within an embodiment.

### TECHNICAL FIELD

The present invention relates to an electronic apparatus for therapeutic or cosmetic use. The invention will more particularly find its application in the so-called electrotherapy field and in particular for grouping together in a channel two families of types of current advantageously combining low frequencies, medium frequencies (electrostimulation) and high frequencies (radio frequency).

### PRIOR ART

Electrotherapy is a non-hazardous non-invasive technique that consists in using electricity for a therapeutic purpose. This technique is recognised for easing pain, strengthening muscle fibres or accelerating healing of biological tissues. Three major families of frequencies exist, such as low frequencies (1 Hz-150 Hz) for superficial neurostimulating action, medium frequencies (1 kHz-10 kHz) for deep neurostimulating action and finally high frequencies (100 kHz-1.2 MHz) for superficial or deep selective diathermal action. Low frequencies (LF) and medium frequencies (MF) are generally designated as electrostimulation and high frequencies (HF) are designated as radio frequencies.

These various types of electrotherapy current circulate between two plates that fulfil the role of electrodes in contact with the skin and are fixed or movable. The various types of current are used sequentially by the practitioner. In low frequency (1 Hz-150 Hz), the current designated “microcurrent” is composed of a string of pulses for treating chronic pain, inflammations, drainage and muscle recruitment. In general, it is characterised by pulses the electrical characteristics of which are appreciably inferior to other forms of electrostimulation current. Microcurrent proves to be particularly indicated for localised and superficial regions.

In medium frequency (1 kHz and 10 kHz), the current designated interferential is of the sinusoidal type modulated by a low-frequency signal and is able to penetrate in depth with good tolerance on the part of the patient. It is composed of a sinusoidal carrier between 1 kHz and 10 kHz with amplitude modulation to create a carried low-frequency signal that makes it possible to contract deep muscles with antalgic action. This current is particularly used to recruit the deep muscles essential to the posture of the patient.

Electrostimulation appliances are therefore known for muscle strengthening, used either autonomously or by a practitioner such as a kinesitherapist.

Moreover, heat is a therapeutic modality that has been used for many years in kinesitherapy and is divided into two categories: superficial heating agents and deep heating agents. The modalities of deep thermotherapy include long-wave and short-wave therapeutic diathermy, ultrasound and contact radio frequency, the latter also being referred to as high-frequency current, which is between 100 kHz and 1.2 MHz. This type of deep thermotherapy is called diathermy.

Tecartherapy is also known, based on TECAR (Transfer of Energy Capacitive And Resistive) current. Tecartherapy is treatment by contact radio frequency. It is a type of electrotherapy that uses high-frequency currents. Development of this technology was enabled by the development of resistive electrodes at the start of the nineties. Tecartherapy generates a contact radio-frequency current in the high-frequency spectrum. In capacitive mode, the resonance will be greater in the soft tissues rather than the muscles while in resistive mode the resonance will be greater in depth in the hard tissues such as ligaments or bones. Conventionally, the therapeutic wavelength spectrum ranges from 300 kHz to 1.2 MHz.

There are therefore numerous technologies in the service of therapeutic treatment by electricity, however, each treatment must be implemented separately with separate appliances, which complicates the treatment protocols and limits access to the various technologies when the practitioner must be equipped with a specific machine for each application.

There is therefore a need to propose a technological solution that allows optimised and simplified treatments.

The other objects, features and advantages of the present invention will appear upon examining the following description and the appended drawings. It is understood that other advantages can be incorporated.

### SUMMARY

To achieve this objective, according to one embodiment, an electrotherapy device is provided comprising a first voltage-generating module configured to generate a first voltage having a first frequency and comprising a first connection terminal and a second connection terminal, and a second voltage-generating module configured to generate a second voltage having a second frequency strictly higher than the first frequency and comprising a third connection terminal and a fourth connection terminal, characterised in that it comprises a transmission channel and a reception channel and in that the first connection terminal and the third connection terminal are connected to said transmission channel and in that the second connection terminal and the fourth connection terminal are connected to said reception channel.

The invention makes it possible to apply two voltages of two different frequencies by one and the same transmission channel and one and the same reception channel.

More particularly, the electrotherapy device comprises a control unit configured to control the first voltage-generating module to generate the first voltage at a first frequency, and to control the second voltage-generating module to generate the second voltage at a second frequency simultaneously.

Advantageously, the control unit is configured to generate in the transmission channel a signal associating a sinusoidal voltage of a first frequency modulated by a third frequency with a sinusoidal voltage of a second frequency modulated by a fourth frequency, the signal being the sum of the voltage of the first generating module and of the voltage of the second generating module.

Another aspect relates to a method for operating an electrotherapy device as described above comprising:
a. generating a first voltage at a first frequency by the first generating module, and
b. generating a second voltage at a second frequency strictly higher than the first frequency by the second generating module, steps a and b being simultaneous. Thus the two voltage generators operate simultaneously.

### BRIEF DESCRIPTION OF THE FIGURES

The aims, purposes, characteristics and advantages of the invention will emerge more clearly from the detailed description of one embodiment thereof, which is illustrated by means of the following accompanying drawings, in which:

FIG. 1 shows the diagram of the electrical circuit of the device according to the invention.

FIG. 2A shows the sinusoidal medium-frequency voltage generated by the first generating module.

FIG. 2B shows the sinusoidal low-frequency unitary signal generated by the first generating module.

FIG. 2C shows the medium-frequency sinusoidal voltage modulated by the low frequencies generated by the first generating module.

FIG. 3A shows the sinusoidal high frequency generated by the second generating module.

FIG. 3B shows the square low-frequency unitary signal generated by the second generating module, more precisely by the activation command.

FIG. 3C shows a high-frequency sinusoidal signal modulated by pulse low frequencies generated by the second generating module.

FIG. 4 shows the medium-frequency sinusoidal signal modulated by low frequencies associated with the high-frequency sinusoidal signal modulated by low frequencies in the transmission channel during a simultaneous application of the two currents.

The drawings are provided by way of example and are not intended to limit the scope of the invention. They constitute outline diagrammatic views intended to facilitate understanding of the invention and are not necessarily to the scale of practical applications.

### DETAILED DESCRIPTION

Before giving a detailed review of embodiments of the invention, optional features are set out below, which can optionally be used as an alternative to or in combination with one another.

According to one example, the first voltage-generating module 100 is configured to generate a sinusoidal voltage.

According to one example, the first frequency is comprised in a first frequency interval between 1 kHz and 10 kHz.

According to one example, the first voltage-generating module 100 is configured to generate a sinusoidal voltage of a first frequency modulated by a third frequency.

According to one example, the third frequency is comprised in a third frequency interval between 1 Hz and 150 Hz.

According to one example, the third frequency is a sinusoidal voltage.

The invention proposes to create a medium-frequency sinusoidal signal in order to modulate the low-frequency signal (electrostimulation) and to combine it with a high-frequency sinusoidal signal (diathermy). The invention makes it possible to combine, within a single channel, the advantages of electrostimulation and of radiofrequency generating diathermy. The invention produces a non-invasive current stimulating the natural healing mechanisms of the body and favouring cell exchange. It offers excellent rehabilitation results by virtue of rapid recovery of the muscle and joint functions.

The result is a sinusoidal current of 1 kHz to 10 kHz modulated at a frequency of 1 Hz to 150 Hz.

In electrostimulation, modulation of the stimulating current makes it possible to avoid tetany of the excited muscles.

According to one example, the second voltage-generating module 200 is configured to generate a sinusoidal voltage.

According to one example, the second frequency is comprised in a second frequency interval between 100 kHz and 10 MHz.

According to one example, the second voltage-generating module 200 is configured to generate a sinusoidal voltage of a second frequency modulated by a fourth frequency.

According to one example, the fourth frequency is comprised in a fourth frequency interval between 1 Hz and 150 Hz.

According to one example, the fourth frequency is of the pulse type.

According to one example, the second voltage-generating module 200 comprises an activation command 38 configured to generate a pulse sinusoidal voltage at the fourth frequency.

According to one example, the first voltage-generating module 100 comprises a transmission command 37 configured to transmit the voltage of the first generating module at the fourth frequency.

According to one example, the first generating module 100 comprises a first transformer 11 having a first inductance and the second generating module 200 comprises a second transformer 21 having a second inductance, preferably the ratio of the first inductance and second inductance being greater than 500, preferentially greater than 1000.

According to one example, the second generating module 200 comprises an output filter 24, advantageously associated with a second transformer 21 comprising an LC circuit comprising a coil 26 (L) and a capacitor 25.

According to one example, the output filter 24 comprises a resonant capacitor 27 for resonating at the second frequency of the second generator.

According to one example, the control unit is configured to vary the impedance in the transmission channel 4 when it controls the first module 100.

According to one example, the method for operating an electrotherapy device 1 in which, when the first generating module 100 is active, the presented impedance of the device varies. Which creates a modulation of the high-frequency sinusoidal voltage with the low-frequency-modulated medium-frequency voltage.

According to one example, the method for operating an electrotherapy device 1 when the first generating module 100 is active, the second transformer 21 is disconnected. Otherwise there would be a risk of having a short-circuit at the output of the first transformer 11 not making it possible to generate an output current.

According to one example, the method for operating an electrotherapy device 1 in which, when the second generating module 200 is active, the first transformer 11 does not modify the sinusoidal current at the output of the second transformer 21. The impedance presented is negligible compared with that of the user 30.

The present invention relates to an electrotherapy device 1 comprising two voltage-generating modules 100, 200. Advantageously, the two voltage-generating modules are isolated.

Advantageously, the device makes it possible to simultaneously combine the medium-frequency interferential currents and the high-frequency tecartherapy currents, preferably with pulses for simulating low-frequency microcurrents.

The device 1 comprises a first voltage-generating module 100 comprising a first voltage generator 10. The first voltage generator 10 is configured to generate a first voltage having a first frequency.

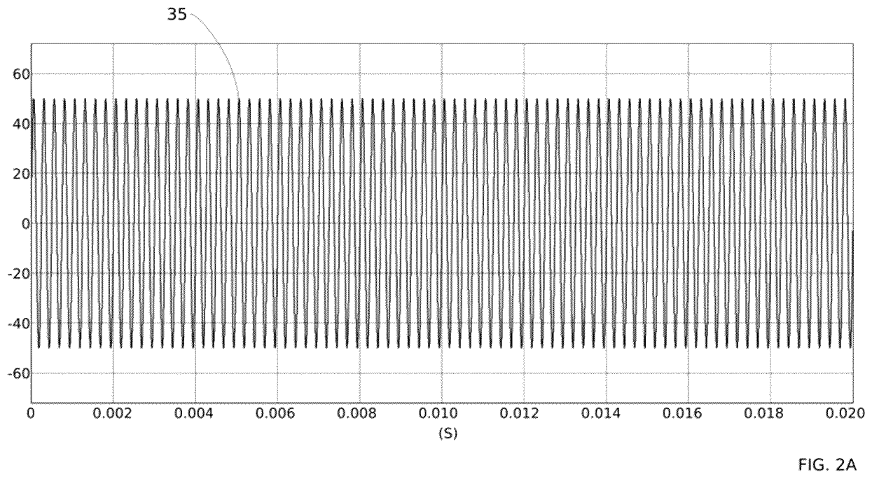

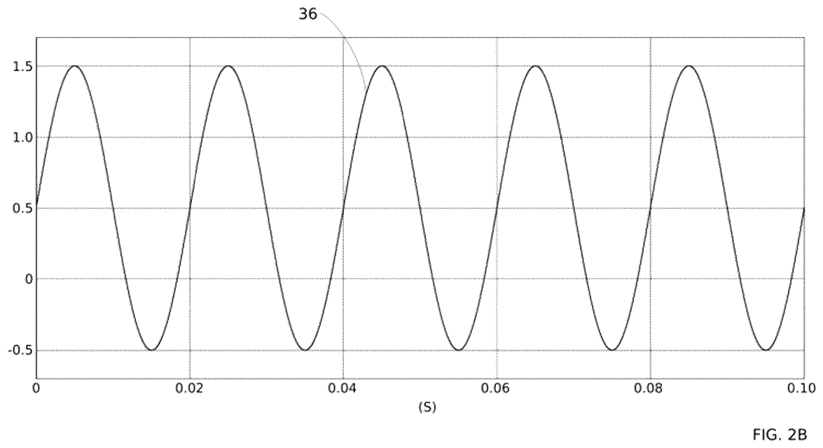

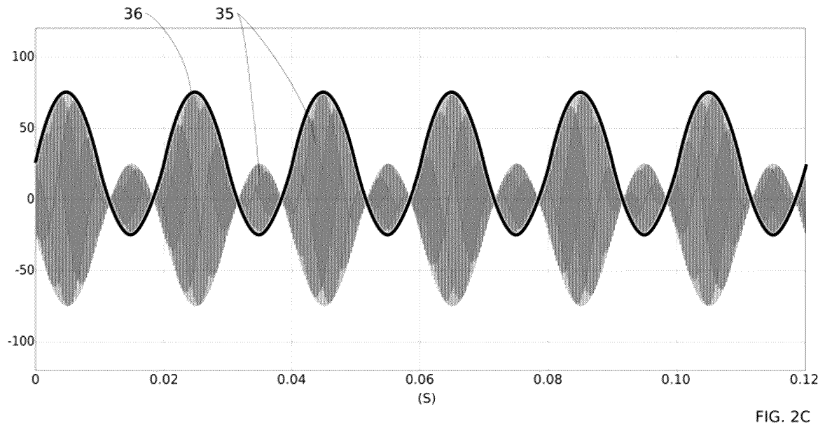

Advantageously, the first voltage is a sinusoidal voltage. Preferentially, the first frequency lies in a first frequency interval between 1 kHz and 10 kHz. This frequency interval means frequencies of the medium-frequency type. An example of sinusoidal medium-frequency voltage is illustrated in FIG. 2A. This frequency interval has in particular a draining effect and participates in muscle recovery. The first voltage-generating module 100 comprises a first connection terminal 12 and a second connection terminal 13. This first generating module 100 is used for electrostimulation applications and/or for in-depth muscle strengthening.

The generator 100 advantageously develops a medium-frequency sinusoidal current between 1 kHz and 10 kHz preferentially with a low-frequency amplitude modulation for stimulating comfortable in-depth muscle recruitment.

According to a preferred embodiment, the device, more precisely the first voltage-generating module 100, comprises a first transformer 11 associated with the first current generator 10.

Advantageously, the first voltage-generating module 100 comprises a first modulator configured to generate a third voltage modulating the first voltage. The third voltage is preferentially a sinusoidal voltage. The first modulator can for example implement a modulation by pulse width modulation (PWM).

The first modulator makes it possible to modulate the first frequency by a third frequency. The third frequency is strictly lower than the first frequency. The third frequency lies in a third frequency interval preferentially being smaller than the first frequency interval. The third frequency interval preferentially lies between 1 Hz and 150 Hz. This frequency interval means frequencies of the low-frequency type.

According to one possibility, the third frequency is variable in the course of operation. During the operation of the first generating module 100, the third frequency can vary in order to vary the modulation of the first frequency according in particular to the requirement of the user. An example of a low-frequency unitary signal is illustrated in FIG. 2B. The signal is shown with a multiplication factor, on the Y axis, between 0 and 1.5. Advantageously, the sinusoid is created directly from the primary division. Pulse width modulation makes it possible to have a sinusoid that can be amplitude modulated at a low frequency, for example of the order of 1 Hz to 150 Hz. The result is a sinusoidal voltage of 1 kHz to 10 kHz amplitude modulated at a frequency of 1 Hz to 150 Hz.

An example of such a modulation is illustrated in FIG. 2C. In electrostimulation, modulation of the stimulating current makes it possible to avoid tetany of the excited muscles.

The first voltage-generating module 100 makes it possible to stimulate comfortable in-depth muscle contractions.

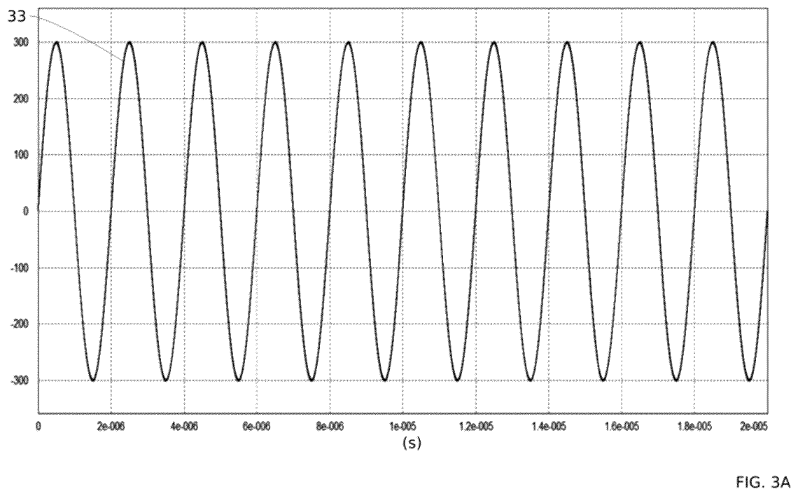

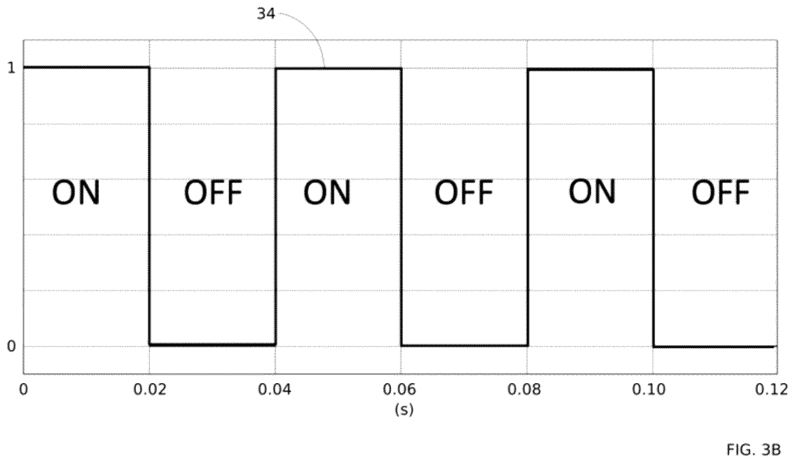

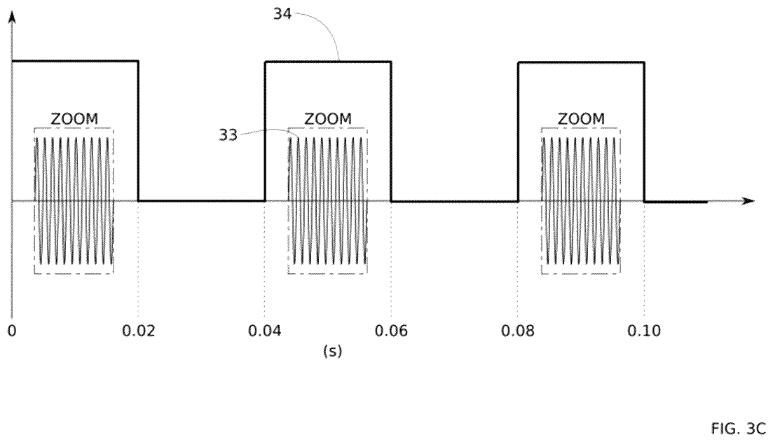

The device 1 comprises a second voltage-generating module 200 comprising a second voltage generator 20. The second voltage generator 20 is configured to generate a second voltage having a second frequency. Advantageously, the second voltage is a sinusoidal voltage. The second frequency is strictly higher than the first frequency. Preferentially, the second frequency lies in a second frequency interval strictly greater than the first interval. Preferentially, the second interval is between 100 kHz and 10 MHz, more precisely from 100 kHz to 1.2 MHz. This frequency interval means frequencies of the high-frequency type. An example of high frequencies is illustrated in FIG. 3A. The second voltage-generating module 200 comprises a third connection terminal 22 and a fourth connection terminal 23. This second generating module 200 is used to have diathermal effects. This type of resultant current advantageously makes it possible to create an acceleration of the metabolism of the biological tissues passed through.

The generator 200 supplies an advantageously sinusoidal high-frequency current between 10 kHz and 10 MHz, more precisely between 100 kHZ and 10 MHz, more specifically from 100 kHz to 1.2 MHz, preferentially with a low-frequency pulse. The high frequency generates a selective diathermal effect and finally the low-frequency creates an anti-inflammatory and analgesic effect.

Advantageously, the second voltage-generating module 200 comprises a command 38 to activate the generator at the second frequency. The activation command 38 is configured to control the output of the generator at the second frequency by a fourth pulse frequency. The fourth frequency is strictly lower than the second frequency. The fourth frequency lies in a fourth frequency interval preferentially being below the second frequency interval. The fourth frequency interval preferentially lies between 1 Hz and 150 Hz. This frequency interval means frequencies of the low-frequency type. An example of a pulse low-frequency is illustrated in FIG. 3B. The fourth frequency is also called a square frequency.

The output voltage of the second voltage-generating module 200 is a high-frequency sinusoidal voltage emitted by low-frequency pulse. An example of a modulation of this type is illustrated in FIG. 3C.

According to a preferred embodiment, the device, more precisely the second voltage-generating module 200, comprises a second transformer 21 associated with the second voltage generator 20.

Advantageously, the device comprises an output filter 24 preferentially associated with the second transformer 21. The output filter 24 makes it possible to filter at the secondary of the second transformer 21. The output filter 24 makes it possible to create a sinusoid.

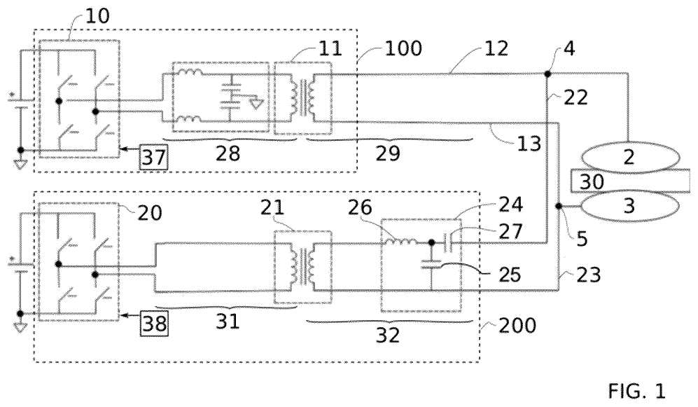

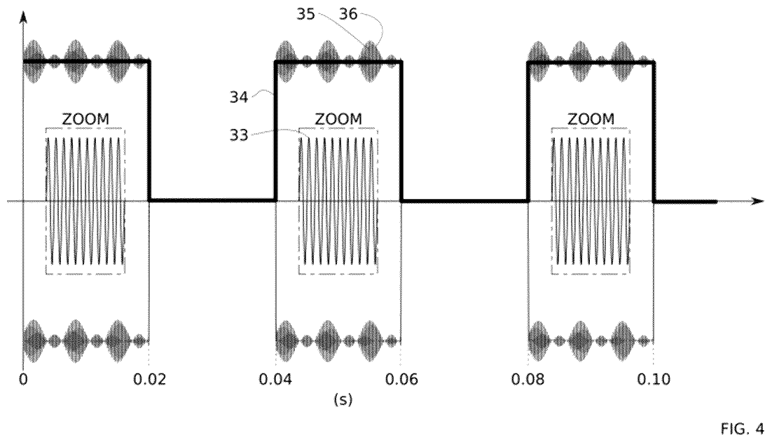

Advantageously, this filter comprises an LC circuit comprising a coil (L) 26 and a capacitor (C) 25. Preferentially, the output filter 24 comprises a resonant capacitor 27 for resonating at the operating frequencies. The resonant capacitor 27 is arranged at the output of the coil 26 and of the capacitor 25. An example of modulation obtained at the output of the output filter 24 according to the electrical circuit illustrated in FIG. 1 is illustrated in FIG. 4. According to this embodiment, the third connection terminal 22 and the fourth connection terminal 23 are arranged at the terminals of the output filter 24.

According to the invention, the device 1 comprises a transmission channel 4 and a reception channel 5. Advantageously, the first terminal 12 and the third terminal 22 are connected to the same transmission channel 4 while the second terminal 13 and the fourth terminal 23 are connected to the same reception channel 5.

Preferentially, the transmission channel 4 is intended to be connected to an electrode, preferentially an active electrode 2. The active electrode 2 can be capacitive and/or resistive.

Preferentially, the reception channel 5 is intended to be connected to an electrode, preferentially a neutral electrode 3. The neutral electrode 3 can be fixed or movable.

The device according to the invention is intended for applying an electrical voltage to the body of a user 30. Advantageously, the active electrode 2 is applied to the body of the user 30 to apply thereto a current while the neutral electrode 3 is also applied to the body of the user 30 to receive the current supplied by the active electrode 2 that passed through a portion of the body of the user 30.

According to one embodiment, the voltage generated by the first generating module 100 is also emitted by pulse at a frequency equal to the fourth frequency generated by the second generating module 200. For this purpose, the first generating module 100 advantageously comprises a command 37 for emitting the output signal of the first generating module 100 that operates by pulse at the same frequency as the fourth frequency of the activation command 38 of the second generating module 200.

The invention enables these two generators 10, 20 to be combined to supply to the transmission channel 4 a low-frequency-modulated medium-frequency sinusoidal current with a high-frequency sinusoidal current as illustrated in FIG. 4.

The invention thus proposes to combine, within a single transmission channel 4, the advantages of electrostimulation on muscle contraction by means of low-frequency currents and the advantages of radio frequency generating diathermy by means of high-frequency currents.

To succeed in mixing the two currents in the same transmission channel 4 without interfering with their quality, the invention proposes to create a medium-frequency current, advantageously sinusoidal, in order to carry the low-frequency signal intended for electrostimulation and to combine it with a high-frequency current, advantageously sinusoidal, preferably of the low-frequency pulse type, intended for diathermy. The invention advantageously produces a non-invasive current stimulating the natural healing mechanisms of the body and favouring cell exchange. It offers excellent rehabilitation results by virtue of rapid recovery of the muscle and joint functions.

The possible applications are a reduction in pain, an elimination of mechanical tensions, a stimulation of blood and lymph circulation and in-depth muscle strengthening and/or acceleration of the natural regeneration of injured or damaged tissues.

According to the invention, the first generating module 100 and the second generating module 200 respectively generate a voltage simultaneously.

Simultaneous means that the first voltage-generating module 100 generates a first voltage transmitted by the transmission channel 4 and, during at least a non-zero given period of time, the second voltage-generating module 200 generates a second voltage transmitted by the same transmission channel 4. During at least this non-zero given period of time, the active electrode 2 supplies the first current and the second current simultaneously, for example to the user 30.

The device comprises a control unit configured to control the generation of the voltage by the first generating module 100 and the generation of the voltage by the second generating module 200.

Advantageously, the control unit is also configured to control the intensity, the voltage and the inductance of the currents generated and/or the impedance of the device.

According to a preferred embodiment, the first transformer 11 has a first inductance and the second transformer 21 has a second inductance, and the ratio of the first inductance and second inductance is advantageously greater than or equal to 500, preferentially greater than or equal to 1000. By way of example, the first transformer 11 is at 590 mH maximum and the second transformer 21 is at 385 μH minimum.

The impedance of the second generating module is negligible compared with that of the user 30.

The main path of the current is thus through the user 30.

When the second generator 20 is active, the first transformer 11 does not modify the sinusoidal voltage of the second transformer 21, because of the inductance, there is no interference of frequencies.

The invention also relates to a method for operating a device according to the invention comprising the following operating steps:
A step a. of generating a first voltage having a first frequency preferentially lying in a first frequency interval by the first voltage-generating module 100.
A step b. of generating a second voltage having a second frequency strictly higher than the second frequency and preferentially lying in a second frequency interval strictly higher than the first frequency interval by the second current-generating module 200.

The method is configured so that steps a and b are simultaneous.

In FIG. 1, the electrical circuit of the device 1 according to the invention comprises a first generating module 100 and a second generating module 200. The first generating module 100 comprises a voltage generator 10 and a first transformer 11. The voltage generator 10 comprises a half-bridge or full-bridge topology for generating a signal. The voltage generator 10 is advantageously a medium-frequency voltage generator controlled by PWM (pulse width modulation). The first generating module 100 comprises a first connection terminal 12 and a second connection terminal 13.

The first transformer 11 is connected to the terminals of the first generator 10. The first transformer 11 comprises, according to a non-limitative embodiment, a first primary circuit 28 advantageously comprising a medium-frequency filter advantageously by PWM and a first secondary circuit 29.

The medium-frequency filter below 10 kHz makes it possible to generate a sinusoid from a square signal. The use of PWM makes it possible to have optimised filtering. The first connection terminal 12 and the second connection terminal 13 are arranged at the secondary circuit 29 of the first transformer 11.

The second generating module 200 comprises a second voltage generator 20 and a second transformer 21. The voltage generator 20 comprises a half-bridge or full-bridge topology for generating a square-wave signal. The second generating module 200 comprises a third connection terminal 22 and a fourth connection terminal 23.

The second transformer 21 is connected to the terminals of the second generator 20. The second transformer 21 comprises, according to a non-limitative embodiment, a second primary circuit 31 and a second secondary circuit 32. Preferentially, as illustrated here, the device comprises an output filter 24 connected to the terminals of the secondary of the second transformer 21. The output filter 24 comprises a coil 26, a resistor 25 and a resonant capacitor 27. The third connection terminal 22 and the fourth connection terminal 23 are arranged on the output filter 24. The output filter is a high-frequency filter. The high-frequency filter above 100 kHz making it possible to generate a sinusoid from a square signal. Advantageously, the structure consisting of coil 26—resistor 25 and a resonant capacitor 27 makes it possible to improve the shape of the sinusoid by resonance preferentially at a frequency f0. By way of preferred example, f0 is set at 400 kHz.

The first terminal 11 and the third terminal 21 are connected to the transmission channel 4 and the second terminal 13 and the fourth terminal 23 are connected to the reception channel 5.

Preferentially, as illustrated, the transmission channel 4 is connected to at least one active electrode 2 and the reception channel 5 is connected to at least one neutral electrode 3. The electrodes 2, 3 are applied to the user 30.

The voltage generator 10 and the voltage generator 20 are isolated.

FIGS. 2A to 2C illustrates the voltage generated by the first generating module 100.

FIG. 2A illustrates the voltage at the first frequency, here 4 kHz, lying in the first frequency interval corresponding to the medium frequencies. The voltage is a sinusoidal voltage, curve 35.

FIG. 2B illustrates the signal modulating at the second frequency, here 50 kHz, lying in the third frequency interval corresponding to the low frequencies. The signal is a sinusoidal signal, curve 36.

The first voltage-generating module 100 thus generates the sinusoidal voltage illustrated in FIG. 2C, having a medium frequency at 4 kHz, curve 35, amplitude modulated at 150% by low frequencies, curve 36. The modulation takes place at 100% and then at 50% by the low-frequency modulation frequency.

FIGS. 3A to 3C illustrate the voltage generated by the second generating module 200.

FIG. 3A illustrates the voltage at the second frequency, here 500 kHz, lying in the second frequency interval corresponding to the high frequencies. The voltage is a sinusoidal voltage, curve 33.

FIG. 3B illustrates the signal modulating at the fourth frequency, here 25 kHz, lying in the fourth frequency interval corresponding to the low frequencies. The signal is a square signal The modulating signal is thus a pulse, curve 34.

The second voltage-generating module 200 thus generates the sinusoidal voltage illustrated in FIG. 3C, having a high frequency at 500 kHz, modulated by low frequencies of the square type.

A detail of a sinusoidal voltage illustrated in FIG. 3A is shown in the squares in FIG. 3C. Thus, the high-frequency voltage that is modulated by the low-frequency pulses is found.

FIG. 4 shows the sinusoidal voltage at the output of the transmission channel 4 during a simultaneous application of two voltages. The sinusoidal voltage obtained is the sum of the first voltage generated by the first generating module 100 and of the second voltage generated by the second generating module 200. The output signal in the transmission channel 4 is a sinusoidal voltage having high frequencies and medium frequencies modulated by low frequencies. Here, the first voltage generated by the first generating module 100 is also modulated by the pulse low frequency of the second generating module 200. For this purpose, as described above, the device comprises a command 37 for transmission of the transmitted voltage configured to transmit the voltage of the first generating module at the fourth frequency. Therefore here, on the low frequency of the pulses (curve 34), the high frequency (curve 33) and the medium frequency (35) modulated by low frequency (curve 36) are found in amplitude greater than 100%.

The invention is not limited to the aforementioned embodiments and includes all the embodiments covered by the application.

### LIST OF REFERENCES

1 Device
2 Active electrode
3 Neutral electrode
4 Transmission channel
5 Reception channel
10 First generator
11 First transformer
12. First connection terminal
13. Second connection terminal
20. Second generator
21. Second transformer
22. Third connection terminal
23. Fourth connection terminal
24. Output filter
25. Capacitor
26. Coil
27. Resonant capacitor
28. First primary circuit
29. First secondary circuit
30. User
31. Second primary circuit
32. Second secondary circuit
33. High-frequency curve
34. Pulse low-frequency curve
35. Medium-frequency carrier curve
36. Low-frequency curve
37. Transmission command
38. Activation command
100. First generating module
200. Second generating module
300. Carrier
400. Application period
500. First period
600. Second period

1. An electrotherapy device comprising:
a first voltage-generating module configured to generate a first voltage having a first frequency and comprising a first connection terminal and a second connection terminal,
a second voltage-generating module configured to generate a second voltage having a second frequency strictly higher than the first frequency and comprising a third connection terminal and a fourth connection terminal,
wherein the device comprises:
a transmission channel,
a reception channel,
and in that the first connection terminal and the third connection terminal are connected to said transmission channel and in that the second connection terminal and the fourth connection terminal are connected to said reception channel, and
a control unit configured to simultaneously:
a. control the first voltage-generating module to generate the first voltage at a first frequency, and
b. control the second voltage-generating module to generate the second voltage at a second frequency,
the control unit being configured to generate in the transmission channel a signal associating a sinusoidal voltage at the first frequency modulated by a third frequency and a sinusoidal voltage at the second frequency modulated by a fourth frequency, the signal being the sum of the voltage of the first generating module and of the voltage of the second generating module.

2. The device according to the preceding claim 1, wherein the first voltage-generating module is configured to generate a sinusoidal voltage.

3. The device according to claim 1, wherein the first frequency is comprised in a first frequency interval between 1 kHz and 10 kHz.

4. The device according to claim 1, wherein the first voltage-generating module is configured to generate a sinusoidal voltage of the first frequency modulated by the third frequency.

5. The device according to claim 1, wherein the third frequency is comprised in a third frequency interval between 1 Hz and 150 Hz.

6. The device according to claim 1, wherein the second voltage-generating module is configured to generate a sinusoidal voltage.

7. The device according to claim 1, wherein the second frequency is comprised in a second frequency interval between 100 kHz and 10 MHz.

8. The device according to claim 1, wherein the second voltage-generating module is configured to generate a sinusoidal voltage of the second frequency modulated by the fourth frequency.

9. The device according to claim 1, wherein the fourth frequency is comprised in a fourth frequency interval between 1 Hz and 150 Hz.

10. The device according to claim 1, wherein the fourth frequency is of the pulse type.

11. The device according to claim 1, wherein the second voltage-generating module comprises an activation command configured to generate a pulse sinusoidal voltage at the fourth frequency.

12. The device according to claim 1, wherein the first voltage-generating module comprises a transmission command configured to transmit the voltage of the first generating module at the fourth frequency.

13. The electrotherapy device according to claim 1, wherein the first generating module comprises a first transformer having a first inductance and the second generating module comprises a second transformer having a second inductance.

14. The electrotherapy device according to claim 1, wherein the second generating module comprises an output filter comprising an LC circuit comprising a coil and a capacitor.

15. The electrotherapy device according to claim 14, wherein the output filter comprises a resonant capacitor for resonating at the second frequency of the second generating module.

16. The electrotherapy device according to claim 1, wherein the control unit is configured to vary the impedance in the transmission channel responsive to the control unit controlling the first voltage-generating module to generate the first voltage at a first frequency.

17. The electrotherapy device according to claim 13 wherein the ratio of the first inductance and second inductance is greater than 500.

18. The electrotherapy device according to claim 13, wherein the ratio of the first inductance and second inductance is greater than 1000.

19. A process for electrotherapy by using the electrotherapy device according to claim 1 comprising the followings steps:
the first voltage generator generates a first voltage having a first frequency and the first voltage generator comprising a first connection terminal and a second connection terminal,
the second voltage generator generates a second voltage having a second frequency strictly higher than the first frequency and the second voltage generator comprising a third connection terminal and a fourth connection terminal,
and the first connection terminal and the third connection terminal are connected to said transmission channel and the second connection terminal and the fourth connection terminal are connected to said reception channel,
the control unit simultaneously:
a. controls the first voltage-generating module to generate the first voltage at a first frequency,
b. controls the second voltage-generating module the second voltage at a second frequency,
the control unit generates in the transmission channel a signal associating a sinusoidal voltage at the first frequency modulated by the third frequency and associated with a sinusoidal voltage at the second frequency modulated by the fourth frequency, the signal being the sum of the voltage of the first generator and of the voltage of the second generator.
